(19) United States
(12) Patent Application Publication
(10) Pub. No.: US 2010/0248344 A1
(43) Pub. Date: Sep. 30, 2010

(54) Methanogenic reactor

(75) Inventors: Bruce G. Schroder, Harrisburg, SD (US); Russell A. Pohl, Sioux Falls, SD (US)

Correspondence Address: Ekse Law Firm, PC, Water Fall Plaza, 6116 S. Lyncrest Avenue, Suite 105, Sioux Falls, SD, 57108, US

(73) Assignee: Tech V, LLC

(21) Appl. No.: 12/383,761

(22) Filed: Mar. 27, 2009

(51) Int. Cl. C12M 1/36; C12M 1/00; C12M 1/02

(52) U.S. Cl. 435/286.1; 435/289.1; 435/296.1; 435/302.1

(57) ABSTRACT

A methanogenic reactor for the production of methane, cellular biomass and other useful products for use in the manufacturing of specialty chemicals. The methanogenic reactor includes a bottom wall, perimeter wall, and top wall defining an interior space environmentally separable from an exterior space outside of the reactor vessel for holding a methanogenic culture and growth media. The reactor also includes at least one sparger positioned substantially within the interior space for facilitating the direction of an input gas stream into the reactor to be brought into contact with the methanogenic culture. The reactor also includes an output material stream port for releasing an output material stream created at least in part by the methanogenic culture.

### REFERENCE TO RELATED APPLICATION

This application is related to application serial number TBD, entitled System For The Production Of Methane And Other Useful Products And Method Of Use filed Mar. 13, 2009.

### BACKGROUND OF THE INVENTION

1. Field of the Invention

The present invention relates to the generation of green natural gas through methanogenic conversion and more particularly pertains to a new methanogenic reactor for generating natural gas and cellular biomass from a variety of input material including syngas, mixed gas, or combined individual gas streams.

2. Description of the Prior Art

The use of methanogens and methanogenic processes is known in the prior art. More specifically, the systems utilizing methanogens to generate natural gas heretofore devised and utilized have generally been either capturing the gaseous output of naturally occurring systems, such as the Volta Experiment on Lake Como in 1778 or anaerobic digestion systems which consist basically of familiar, expected and obvious biological, chemical, and structural configurations, notwithstanding the myriad of designs encompassed by the crowded prior art which have been developed for the fulfillment of countless objectives and requirements.

The process of Methanogenesis is fairly well known. The following references provide a good working overview of the methanogenic process and are hereby incorporated by reference for all purposes: Archea: Molecular and Cellular Biology—Chapter 13 Methanogenesis, James G. Ferry and Kyle A. Kastead, Department of Biochemestry and Molecular Biology, The Pennsylvania State University, University Park, Pa., edited by Ricardo Cavicchiolo, ©2007 ASM Press, Washington, DC; and Continuous Cultures Limited by a Gaseous Substrate: Development of a Simple, unstructured Mathematical Model and Experimental Verification with Methanobacterium thermoautotrophicum, N. Schill; W. M. van Gulik, D. Voisard, and U. von Stockar, Institute of Chemical Engineering, Swiss Federal Institute of Technology, Lausanne (EPFL), CH-1015 Lausanne, Switzerland, Biotechnology and Bioengineering, Vol. 51, P6450658 (1996) John Wiley & Sons, Inc.

Illustrative examples of the types of systems known in the prior art include anaerobic digestion systems and U.S. Pat. Nos. 1,940,944; 2,097,454; 3,640,846; 4,722,741; 5,821,111 and application no. PCT/US07/71138.

In these respects, the methanogenic reactor according to the present invention substantially departs from the conventional concepts and designs of the prior art, and in so doing provides an apparatus primarily developed for the purpose of generating green natural gas and cellular biomass from a variety of source materials.

### SUMMARY OF THE INVENTION

To promote the development of renewable energy sources, the United States government has identified a “billion ton” goal of biomass production per year. At present the largest single component of that supply is corn stover. It is widely accepted that adverse ecological effects of corn production such as the anoxic zone in the Caribbean will encourage biomass production from other sources. Chief among these alternatives will be perennial grasses such as switchgrass and native prairie grasses. Testing currently underway on novel, high yield grasses such as miscanthus points to the prospect of biomass production in lieu of conventional crops on marginal lands.

Despite these developments on the production side there remain critical issues on the conversion of these biomass sources to useable forms as substitutes for fossil fuels.

Pelletizing improves the handling characteristics of biomass, but adds enough cost to the resulting fuel cost to largely eliminate any fuel cost advantage. In addition, biomass fuels burn dirty, producing sulfur and nitrogen oxides and hydrogen chloride. Equipment to burn these fuels is expensive and air permitting remains problematic. A clean solution to these limitations would be to convert biomass into pipeline quality biomethane near the point of origin for transmission to existing natural gas customers via existing natural gas pipelines. This same process can also supply biomethane to specialty chemical facilities for the production of green specialty chemicals, including but not limited to “green plastics”. Further, the present invention also generates cellular biomass, which may be utilized as a food or nutrient for livestock and humans

The two primary routes to biomethane currently recognized are anaerobic digestion and thermochemical conversion. A third process for the conversion of biomass to liquid fuels is being pursued which involves enzymatic breakdown of cellulose and hemicelluloses into fermentable sugars. While these processes are effective on some feedstocks and at some capacities, none of them provide a fully satisfactory route to biomass use.

To understand why this is so, it is helpful to understand the progression of plant composition during the growing season. The three primary structures in a plant are cellulose, hemicelluloses, and lignin. These compounds are in turn polymers of 6-carbon sugars, 5-carbon sugars, and phenolics respectively. As the plant matures, there is a progressive conversion of cellulose and hemicelluloses to lignin. This is reflected in the decrease of total digestable nutrients and the increase of acid detergent fiber content.

Anaerobic digestion uses mixed cultures of microbes to break down biomass into fermentable sugars, amino acids, and organic acids. This process is multi-step and is subject to upset by over-production of organic acids which kill the methanogenic organisims. The great benefit of anaerobic digestion is that it is generally recognized as specific, producing methane and carbon dioxide in a readily recoverable form.

Anaerobic digestion of grasses and corn stover has been extensively studied. Mahert (Mahert, Pia, et al, “Batch and Semi-continuous Biogas Production from Different Grass Species”. December 2005) and others have studied the potential for biomethane production from various grasses. The chief finding of this work is that while biomethane can be produced by this route, the required digester volume per unit of energy produced is uneconomical. In addition, the grasses must be harvested at or before full bloom. Corn stover is substantially limited and in some instances near impervious to anaerobic digestion.

A further disadvantage of anaerobic digestion of grasses is that when native or prairie grass is cut before October, the yield the following year is half or less of what is expected. This appears to be related to the manner in which nutrients are returned to the root structure after frost.

Thermochemcial conversion of biomass to biomethane and liquid fuels is a proven technology base on some old coal chemistry. A large scale coal to natural gas plant at Beulah, N.D. has been in operation since the late 1980's. The chief limitation of this technology is that it strongly favors large scale operations, generally over 400 tons per day.

Enzymatic processes to break down biomass to fermentable sugars remain an elusive and expensive undertaking. Even if successful, however, enzymatic processes are likely to be highly specific to certain species and perhaps even varieties within species due to their high specificity. One of the objectives associated with biomass production is promotion of multiple species cultivation. Highly specific enzyme processes will tend to promote monocultures and leave the ecosystem no richer than a corn/soybean mix.

As the foregoing shows, there is room for development of a novel process which will address the limitations of all the current options. Such a process will have at least some of the following characteristics:
1) It will produce a fuel which is directly compatible with existing energy distribution and use equipment;
2) It will use a variety of feed stocks ranging from corn stover to perennial grasses to wood without loss of yield per ton of saleable energy;
3) It will utilize feedstock harvested late in season and preferable after frost;
4) It will be economical at a scale of 200 ton per day or less;
5) It will be modular to allow initial construction and expansion as the biomass supply chain becomes established and more efficient and
6) It will produce cellular biomass that can have useful and economic value.

The present invention provides each of these advantages by using a hybrid process which combines the flexibility and power of gasification with the specificity of anaerobic digestion, and with improved efficiency and higher production rates than anaerobic digestion. The gasification step overcomes biomass species and variety variations producing uniform, readily fermentable feedstock to the methanogenic reactor. The culture in the methanogenic reactor is efficient and specific producing only methane, cellular biomass, and water as its co-products.

The present invention utilizes gases which maybe derived from a wide variety of feedstocks ranging from crop residues, low value co-products from agriculture processing and energy crops such as switchgrass and corn stover, waste wood products, and other similar biomass sources. The raw materials may be processed such as being reduced to a uniform size and moisture content (preferably very low) prior to gasification. The gassification process converts the biomass into an intermediate gas stream known as syngas or synthesis gas. The syngas, after going through a heat recovery process, may be directed through a filtering system and or a water gas shift prior to being directed into the methanogenic reactor vessel for conversion by the methanogenic culture into methane.

It is important to note that while the present invention is directed towards providing green natural gas from biomass, the same process can be done with municipal or landfill wastes or nonconventional carbon and hydrogen sources (collectively “landfill waste”). The use of the present invention with landfill wastes as the feed stock would allow the reclamation of hundreds of thousands of acres currently used as landfills. If landfill wastes are utilized as a feedstock, the filtering and cleanup process after gasification can be much more complex than that required for biomass feedstock.

Further, it should be noted that the present invention can be utilized for the generation of cellular biomass. Typical algae systems used for biomass generation produce between 0.2 and 4.0 grams per liter of reactor volume per day. Other methanogenic systems have been reported to produce up to 2.88 grams per liter of reactor volume per day. The present methanogenic reactor, when properly operated in a biomass production mode, can produce 12 grams per liter of reactor volume per hour. This present approximately a 1000× improvement over algae generation systems currently in use.

### BRIEF DESCRIPTION OF THE DRAWINGS

The invention will be better understood and objects of the invention will become apparent when consideration is given to the following detailed description thereof. Such description makes reference to the annexed drawings wherein:

FIG. 1 is a schematic functional block diagram of a new Methanogenic Reactor in use according to the present invention.

FIG. 2 is a schematic functional block diagram of an embodiment of the present invention.

FIG. 3 is a schematic functional block diagram of another embodiment of the present invention.

FIG. 4 is a schematic functional block diagram of a further embodiment of the present invention.

FIG. 5 is a schematic functional block diagram of still a further embodiment of the present invention.

FIG. 6 is a schematic flow diagram of the present invention.

### DESCRIPTION OF PREFERRED EMBODIMENTS

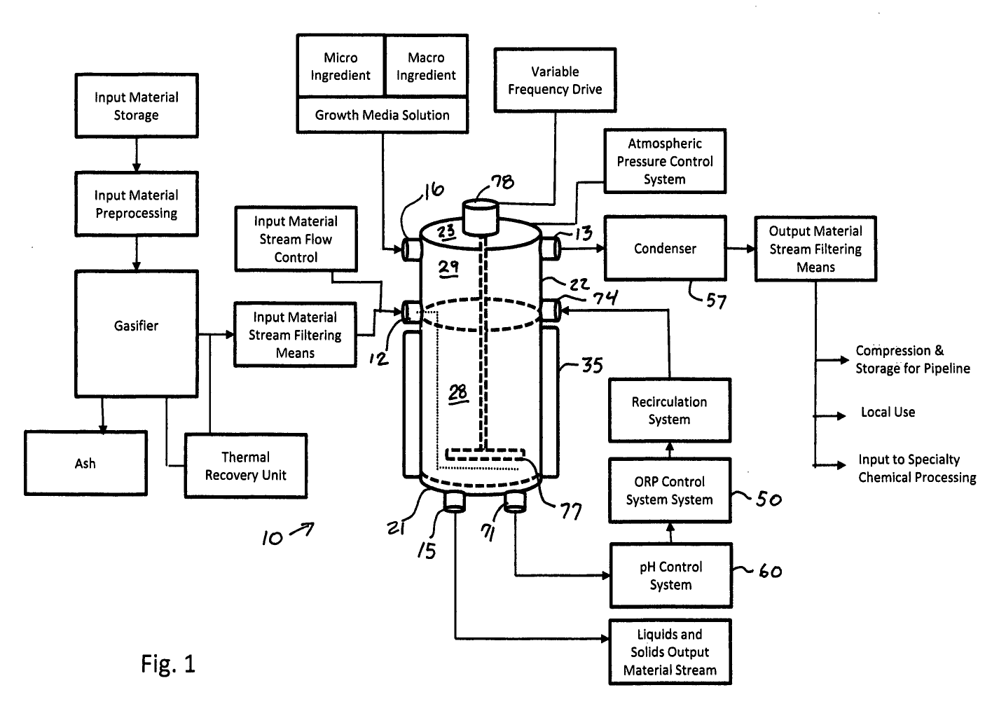

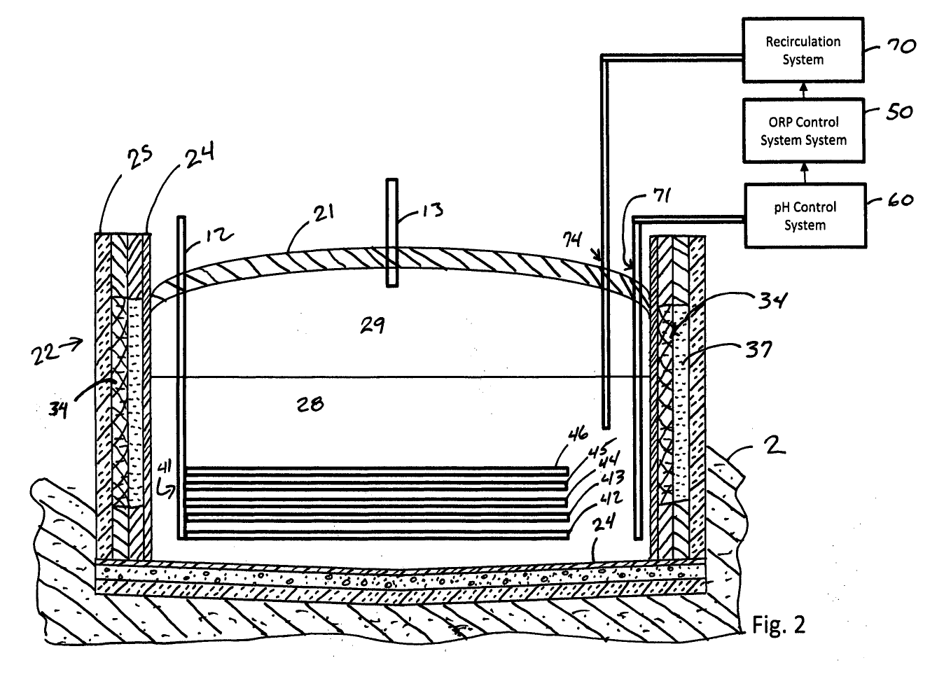

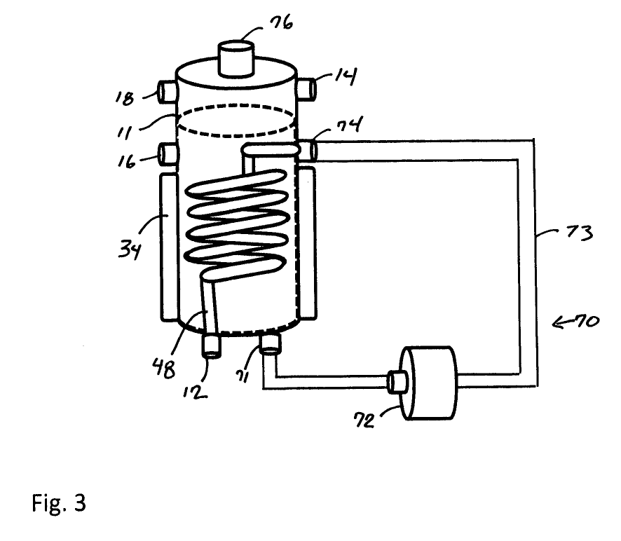

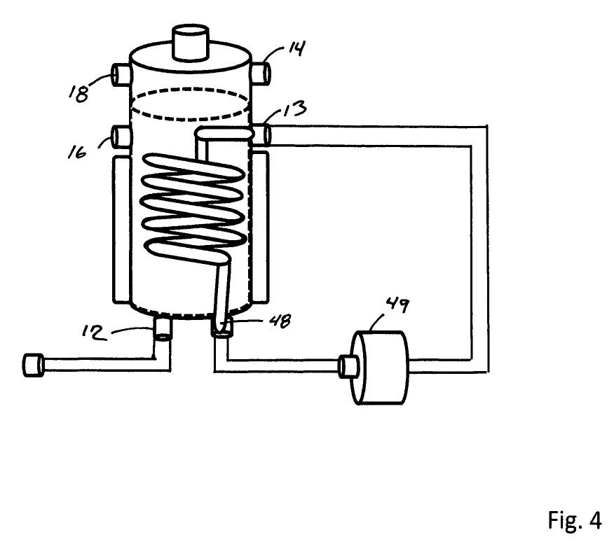

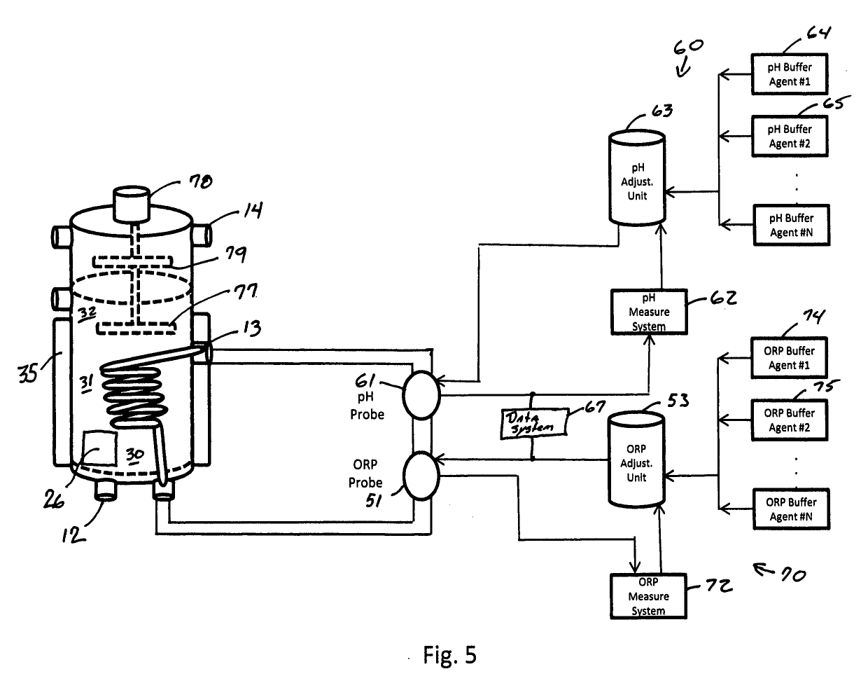

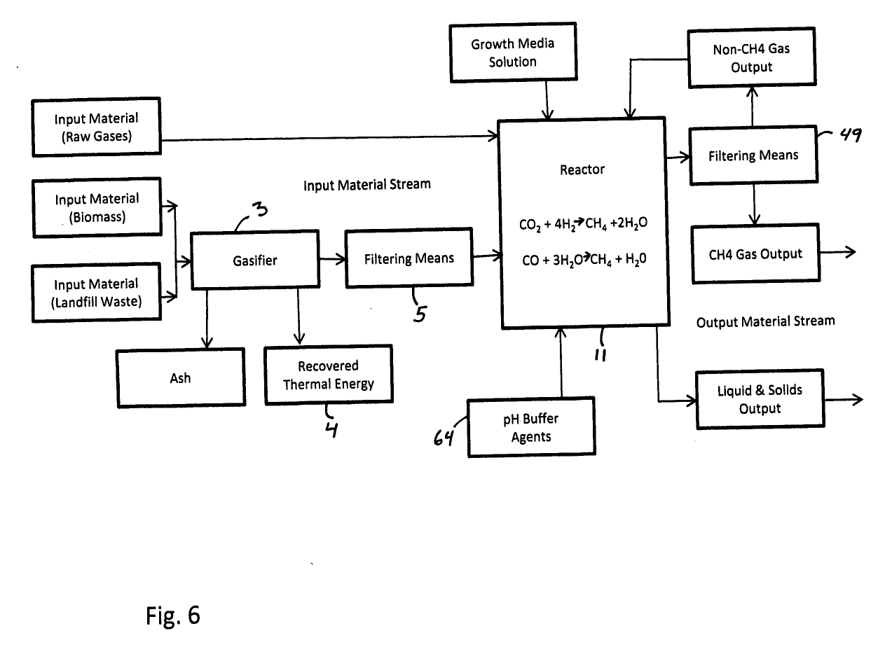

With reference now to the drawings, and in particular to FIGS. 1 through 6 thereof, a new Methanogenic Reactor embodying the principles and concepts of the present invention and generally designated by the reference numeral 10 will be described.

As best illustrated in FIGS. 1 through 6, the methanogenic reactor 10 generally comprises a reactor vessel 11 for the methanogenic conversion of an input material stream to an output materials stream, including a bottom wall 21, perimeter wall 22, and top wall 23. Preferably, the perimeter wall 22 is operationally coupled to the bottom wall 21 and extends upwardly from the bottom wall 21. Similarly, the top wall 23 is operationally coupled to the perimeter wall 22. Thus, the bottom wall 21, perimeter wall 22, and top wall 23 define an interior space 27 which is environmentally separable from an exterior space outside of the reactor vessel 11.

The bottom wall 21 and the perimeter wall 22 may be made out of any suitable material such as stainless steel, fiberglass, or concrete. Additionally, the bottom and perimeter walls 22 may have an interior surface lining 24 of epoxy, a polymeric material, or fiberglass. Preferably, the bottom wall 21 and the perimeter wall 22 are constructed out of the same material for ease of production. However at least one embodiment of the present invention contemplates the bottom wall 21 and the perimeter wall 22 being made out of different materials.

Similarly, the top wall 23 may also made out of any suitable material such as stainless steel, fiberglass, or concrete; and may have an interior surface lines with epoxy, a polymeric material, or fiberglass. However, it should be noted that the top wall 23 may be made out of a different material that the bottom wall 21 or the perimeter wall 22. The top wall 23 may be configured as a floating roof.

At least one embodiment of the reactor vessel 11 is formed substantially in the shape of a sphere, which has a bottom portion, a perimeter portion, and a top portion each corresponding to a bottom wall, perimeter wall and top wall respectively.

In an embodiment the bottom wall 21 has a slope from a back side downwardly to a front side. Preferably, the slope is between 0.075 and 1.5 inches per linear foot.

In another embodiment the bottom wall 21 has a slope from a perimeter downwardly towards a central portion. Preferably, the slope is between 0.75 and 1.5 inches per linear foot.

The present invention contemplates at least one embodiment, in which at least a portion of the reactor vessel 11 abuts an earthen wall 2, such as when at least a portion of the reactor vessel 11 is buried. In such an embodiment, the reactor vessel 11 may also include an insulating layer 25 which abuts the earthen wall 2 and provides a thermal insulation between the reactor vessel 11 and the earthen wall 2.

The reactor vessel 11 may also include an access port 26 for facilitating the clean-out and/or repair of the reactor vessel 11. The access port 26 may be located in the top wall 23, but more preferably is located in the bottom wall 21 or perimeter wall 22.

In a further embodiment, the reactor vessel 11 also includes a thermal conditioning unit 34, which has a thermal transfer portion 35 operationally coupled to the perimeter wall 22. The thermal transfer portion may include either a fluid jacket or an electrical heating coil, which encompasses at least a portion of the perimeter wall 22.

In still a further embodiment, the reactor vessel 11 also includes a culture conditioning chamber 37. The culture conditioning chamber 37 is environmentally coupleable with the interior space 27 and is operationally coupled to the thermal transfer portion 35. The culture conditioning chamber 37 may be used for thermally preconditioning a quantity of culture and media prior to introducing the culture and media into the interior space 27 of the reactor vessel 11.

The reactor vessel 11 may also include at least one sparger 40, positioned substantially within the interior space 27. The sparger 40 is operationally coupled to an input gas stream. When a single sparger 40 is utilized, either a mixed gas must be used as the input gas stream or a mixing assembly may be used to mix various gases from various prior to being introduced into the interior space 27.

In an embodiment, an array of spargers 41 is used. Each one of the array of spargers 41 is operationally coupled to an associated input gas stream. The array of spargers 41 may include at least one of each of the following: a Carbon Dioxide (CO2) sparger 42, a Hydrogen (H2) sparger 43, a Hydrogen Sulfide (H2S) sparger 44, a Carbon Monoxide (CO) sparger 45, and/or a Nitrogen (N) Sparger 46.

In at least one embodiment at least one H2 sparger 43 is positioned vertically above at least one CO2 sparger 42, and at least one H2S sparger 44 is positioned vertically above at least one H2 sparger 43 and at least one CO sparger 45 is positioned vertically above at least one H2S sparger 44.

The Nitrogen sparger 45 can be particularly useful when the reactor is used at least partially for the creation of biomass. The biomass created in the reactor vessel 11 during normal operation may range of approximately 12 grams per liter of effective reactor volume per hour.

Any one of the spargers may be a ring-type sparger, a bayonet type sparger, or any other appropriate configuration. Preferably the spargers create bubbles approximately 1 to 10 microns in diameter.

In at least one embodiment, the reactor vessel 11 also includes an oxidation reduction potential (ORP) control system 50.

In a further embodiment, the oxidation reduction potential control system 50 further includes an oxidation reduction potential probe 51, oxidation reduction potential measurement unit 52, and an oxidation reduction potential adjustment unit 53. Preferably the oxidation reduction potential probe 51 is positioned at least partially within the interior space 27 or a culture/media recycling tube. The oxidation reduction potential probe 51 measures an oxidation reduction potential of a culture/media solution positioned in the interior portion. The oxidation reduction potential measurement unit 52 is operationally coupled to the oxidation reduction potential probe 51 and compares an output of the oxidation reduction potential probe 51 to a predetermined ORP upper value and/or a predetermined ORP lower value. The oxidation reduction potential adjustment unit 53 injects a first oxidation reduction buffer agent 54 into the interior space 27 when the oxidation reduction potential measurement unit 52 determines the output of the oxidation reduction potential probe 51 is at least trending towards the predetermined ORP upper value. Similarly, the oxidation reduction potential adjustment unit 53 injects a second oxidation reduction buffer agent 55 into the interior space 27 when the oxidation reduction potential measurement unit 52 determines the output of the oxidation reduction potential probe 51 is at least trending towards the predetermined ORP lower value.

In a further embodiment the first oxidation reduction buffer agent 54 is either H2S or H2. The ORP upper value is between −400 and −600 mV and more preferably, is approximately −500 mV.

In still a further embodiment the second oxidation reduction buffer agent 55 is CO. The ORP lower value is between −600 and −800 mV and more preferably is approximately −700 mV.

In at least one embodiment the reactor vessel 11 also includes a pH control system 60.

In a further embodiment the pH control system 60 further includes a pH probe 61, a pH measurement unit 62, and a pH adjustment unit 63. Preferably, the pH probe 61 is positioned at least partially within the interior space 27 or a culture/media recycling tube 73, and measures a pH of a culture/media solution positioned in the interior portion. The pH measurement unit 62 is operationally coupled to the pH probe 61 and compares an output of the pH probe 61 to a predetermined pH upper value and/or a predetermined pH lower value. The pH adjustment unit 63 injects a pH buffer agent 64 into the interior space 27 when the pH measurement unit 62 determines the output of the pH probe 61 is at least trending towards the predetermined pH upper value. Similarly, the pH adjustment unit 63 injects a second pH buffer agent 65 into the interior space 27 when the pH measurement unit 62 determines the output of the pH probe 61 is at least trending towards the predetermined pH lower value.

In still a further embodiment the first pH buffer agent 64 includes CO2, and the pH upper value is between 7.5 and 9, and more preferably the pH upper value is approximately 8.

In even still a further embodiment the second pH buffer agent 65 is selected from a group of agents including sodium hydroxide, potassium hydroxide, calcium hydroxide, sodium bicarbonate, ammonia, ammonium and ammonium nitrate. Preferably, the pH lower value is between 6 and 8, and more preferably is approximately 7.6.

In yet a further embodiment, the second buffer agent 65 is selected at least in part based upon the rate of change of the pH of the culture/media in the interior space 27 of the reactor vessel 11.

In at least one embodiment, the reactor vessel 11 also includes an agitation system 76.

In an embodiment, the agitation system 76 further includes an agitation drive means and an impeller 77. The impeller 77 operationally coupled to the agitation drive means, the impeller 77 positioned within the reactor vessel 11. As an illustrative example of an agitation drive means as contemplated by the present invention, a motor electrically coupled to a variable frequency drive to control the speed of the motor may be magnetically coupled to an agitation shaft positioned within the reactor. The impeller 77 is thus operationally coupled to the motor.

In a further embodiment the impeller 77 rotates at between 1100 and 2100 rpm.

In still a further embodiment the impeller 77 rotates at between 1500 and 1800 rpm.

In still yet a further embodiment the impeller 77 rotates at greater than 110% of the resonance of the reactor vessel 11. Resonance being defined as the sympathetic frequency of vibration for the reactor vessel 11.

The impeller 77 may be any physical configuration appropriate for the form factor of the interior space 27 of the reactor vessel 11. Preferably the impeller 77 is a rushton impeller.

The magnetic coupling unit 78 may be operationally coupled to the top wall 23 or the perimeter wall 22.

In at least one embodiment the interior space 27 further includes a culture/media holding space 28 and a head space 29.

In a further embodiment a volume of the culture/media holding space 28 to a volume of the head space 29 has a ratio between 1.5:1 to 5:1.

In a more preferred embodiment a volume of the culture/media holding space 28 to a volume of the head space 29 has a ratio of approximately 2.57:1.

In an embodiment the reactor vessel 11 has an overall interior height between 10 and 220 feet.

In another embodiment the reactor vessel 11 has an overall interior height between 60 and 150 feet.

In a preferred embodiment the reactor vessel 11 has an overall interior height of approximately 140 feet.

In an embodiment the reactor vessel 11 has an overall interior volume between 75000 and 300000 gallons.

In a preferred embodiment the reactor vessel 11 has an overall interior volume of 250000 gallons, with an overall interior height of approximately 140 feet, and a volume of the culture/media holding space 28 to a volume of the head space 29 has a ratio of approximately between 2.3:1 and 2.8:1.

In at least one embodiment, the reactor vessel 11 further includes a culture/media recirculating system 70.

In a further embodiment the culture/media recirculating system 70 further comprising a recirculation output port 71, a recirculation pump 72, a recirculation tube 73, and a recirculation input port 74. The recirculation output port 71 is environmentally coupled to the interior space 27. The recirculation pump 72 operationally coupled to the recirculation output port 71. The recirculation input port 74 is environmentally coupled to the interior space 27 and operationally coupled to the recirculation pump 72.

The reactor vessel 11 may include a plurality of ports for facilitating routing of materials into and out of the interior space 27 of the reactor vessel 11. This plurality of ports may include an input material stream port 12, an output material stream port 13, biomass removal port 15, culture/media input port 16, and/or a culture sampling port 17. Some of these ports may be environmentally coupled to the interior space 27 through a recycling tube 73 or other intermediate structure.

The reactor vessel 11 may also include a secondary vessel 57 environmentally coupled to the output material stream port 13.

In an embodiment, the secondary vessel 57 is a condenser. Preferably the condenser has a cooling jacket 58 to facilitate the removal of moisture and/or foam from the output material stream. Such moisture and/or foam may be returned into the interior space 27 of the reactor vessel 11 or disposed of through a drain port 59 in the secondary vessel 57.

Alternatively, the secondary vessel 57 may be a non-thermal separation system, such as a reverse osmosis system.

In an embodiment the reactor vessel 11 further includes a data system 67 operationally coupled to at least one of the culture media recirculation system 70, oxidation reduction potential control system 50, pH control system 60, agitation system 76, thermal conditioning unit 34, or at least one sparger 40.

In an embodiment the reactor vessel 11 further includes a hydrogen diffuser 48 system positioned substantially within the interior space 27 for releasing hydrogen from a mixed gas stream flowing through the hydrogen diffuser 48 system into the interior space 27.

In a further embodiment the hydrogen diffuser 48 system is operationally coupled between an input material stream port 12 and a first output material stream port 13.

In an alternate embodiment the first output material stream port 13 is operationally coupled to an input of an intermediate processing unit 49 providing a filtering function. The intermediate processing unit preferably includes an output operationally coupled to a second input material stream port 12 environmentally coupled to the interior space 27. Illustrative examples of intermediate processing include filtering means such as PSA and water-gas shift.

In still another alternate embodiment the hydrogen diffuser 48 system is operationally coupled between a first output material stream port 13 and a second output material stream port 14.

In a further embodiment the hydrogen diffuser 48 system further includes a length of tubing having a perimeter wall 22 permeable by hydrogen and substantially impermeable to other components in the mixed gas stream.

The present invention also contemplates the interior space 27 of the reactor vessel 11 having a plurality of zones.

In an embodiment the plurality of zones includes a tank pressure zone 30 having a greater pressure due to the column of culture/media within and above the tank pressure zone 30.

In a further embodiment the plurality of zones includes a diffuser zone 31, and the hydrogen diffuser 48 system is positioned substantially within the diffuser zone 31.

In still a further embodiment the plurality of zones includes an agitation and defoaming zone 32.

Preferably, the diffuser zone 31 is positioned vertically above the tank pressure zone 30 and the agitation and defoaming zone 32 is positioned vertically above the diffuser zone 31.

In an embodiment the interior space 27 is designed for holding a vertical column of media/culture of at least 50 feet.

In more preferred embodiment the interior space 27 is designed for holding a vertical column of media/culture of at least 100 feet.

The reactor vessel 11 may include a defoaming bar 79 positioned substantially within the agitation and defoaming zone 32. Additionally, a defoaming agent input port 18 may be environmentally coupled with the interior space 27 for the selective introduction of a defoaming agent into the interior space 27.

We claim:

1. A reactor vessel for the methanogenic conversion of an input material stream to an output materials stream, comprising:
a bottom wall;
a perimeter wall operationally coupled to said bottom wall and extending upwardly from said bottom wall;
a top wall operationally coupled to said perimeter wall; and
said bottom wall, perimeter wall, and top wall defining an interior space environmentally separable from an exterior space outside of said reactor vessel.

2. The reactor vessel of claim 1, wherein said bottom wall and said perimeter wall comprise stainless steel.

3. The reactor vessel of claim 1, wherein said bottom wall and said perimeter wall comprise concrete.

4. The reactor vessel of claim 3, wherein said bottom wall and said perimeter wall have an interior surface lined with epoxy.

5. The reactor vessel of claim 3, wherein said bottom wall and said perimeter wall have an interior surface lined with a polymeric material.

6. The reactor vessel of claim 3, wherein said bottom wall and said perimeter wall have an interior surface lined with fiberglass.

7. The reactor vessel of claim 1, wherein said bottom wall and said perimeter wall comprise fiberglass.

8. The reactor vessel of claim 1, wherein said bottom wall has a slope from a back side downwardly to a front side.

9. The reactor vessel of claim 8, wherein said slope is between 0.075 and 1.5 inches per linear foot.

10. The reactor vessel of claim 1, wherein said bottom wall has a slope from a perimeter downwardly towards a central portion.

11. The reactor vessel of claim 10, wherein said slope is between 0.75 and 1.5 inches per linear foot.

12. The reactor vessel of claim 1, further comprising an access port located substantially in said bottom wall.

13. The reactor vessel of claim 1, further comprising an access port located substantially in said perimeter wall.

14. The reactor vessel of claim 1, further comprising a thermal conditioning unit, said thermal conditioning unit having a thermal transfer portion operationally coupled to said perimeter wall.

15. The reactor vessel of claim 14, wherein said thermal transfer portion further comprises a fluid jacket encompassing at least a portion of said perimeter wall.

16. The reactor vessel of claim 14, wherein said thermal transfer portion further comprises at least one electrical coil.

17. The reactor vessel of claim 14, further comprising a culture conditioning chamber, said culture conditioning chamber being environmentally coupleable with said interior space, said culture conditioning chamber being operationally coupled to said thermal transfer portion.

18. The reactor vessel of claim 1, further comprising at least one sparger, said being positioned substantially within said interior space, said at least one sparger being operationally coupled to an input gas stream.

19. The reactor vessel of claim 1, further comprising an array of spargers, each one of said array of spargers being operationally coupled to an associated input gas stream.

20. The reactor vessel of claim 19, wherein said array of spargers further comprises at least one CO2 sparger positioned substantially within said interior space, said at least one CO2 sparger being operationally coupled to a CO2 input gas stream.

21. The reactor vessel of claim 19, wherein said array of spargers further comprises at least one H2 sparger positioned substantially within said interior space, said at least one H2 sparger being operationally coupled to an H2 input gas stream.

22. The reactor vessel of claim 19, wherein said array of spargers further comprises at least one H2S sparger positioned substantially within said interior space, said at least one H2S sparger being operationally coupled to an H2S input gas stream.

23. The reactor vessel of claim 19, wherein said array of spargers further comprises at least one CO sparger positioned substantially within said interior space, said at least one CO sparger being operationally coupled to an CO input gas stream.

24. The reactor vessel of claim 19, wherein said array of spargers further comprises at least one nitrogen sparger positioned substantially within said interior space, said at least one nitrogen sparger being operationally coupled to a nitrogen input gas stream.

25. The reactor vessel of claim 19, wherein said array of spargers further comprises:
at least one CO2 sparger positioned substantially within said interior space, said at least one CO2 sparger being operationally coupled to a CO2 input gas stream;
at least one H2 sparger positioned substantially within said interior space, said at least one H2 sparger being operationally coupled to an H2 input gas stream;
at least one H2S sparger positioned substantially within said interior space, said at least one H2S sparger being operationally coupled to an H2S input gas stream; and
at least one CO sparger positioned substantially within said interior space, said at least one CO sparger being operationally coupled to an CO input gas stream.

26. The reactor vessel of claim 25, wherein at least one H2 sparger is positioned vertically above at least one CO2 sparger, and at least one H2S sparger is positioned vertically above at least one H2 sparger and at least one CO sparger is positioned vertically above at least one H2S sparger.

27. The reactor vessel of claim 18, wherein said at least one sparger is a ring-type sparger.

28. The reactor vessel of claim 18, wherein said at least one sparger is a bayonet-type sparger.

29. The reactor vessel of claim 18, wherein said at least one sparger creates bubbles approximately 1 to 10 microns in diameter.

30. The reactor vessel of claim 1, further comprising an oxidation reduction potential control system.

31. The reactor vessel of claim 30, wherein said oxidation reduction potential control system further comprises:
an oxidation reduction potential probe positioned at least partially within said interior space, said oxidation reduction potential probe measuring an oxidation reduction potential of a culture/media solution positioned in said interior portion;
an oxidation reduction potential measurement unit operationally coupled to said oxidation reduction potential probe, said oxidation reduction potential measurement unit comparing an output of said oxidation reduction potential probe to a predetermined ORP upper value and a predetermined ORP lower value; and
an oxidation reduction potential adjustment unit, said oxidation reduction potential adjustment unit injecting a first oxidation reduction buffer agent into said interior space when said oxidation reduction potential measurement unit determines said output of said oxidation reduction potential probe is at least trending towards said predetermined ORP upper value, said oxidation reduction potential adjustment unit injecting a second oxidation reduction buffer agent into said interior space when said oxidation reduction potential measurement unit determines said output of said oxidation reduction potential probe is at least trending towards said predetermined ORP lower value.

32. The reactor vessel of claim 31, wherein said first oxidation reduction buffer agent comprises H2S.

33. The reactor vessel of claim 31, wherein said first oxidation reduction buffer agent comprises H2.

34. The reactor vessel of claim 31, wherein said ORP upper value is between −400 and −600 mV.

35. The reactor vessel of claim 31, wherein said ORP upper value is approximately −500 mV.

36. The reactor vessel of claim 31, wherein said second oxidation reduction buffer agent comprises CO.

37. The reactor vessel of claim 31, wherein said ORP lower value is between −600 and −800 mV.

38. The reactor vessel of claim 31, wherein said ORP lower value is −700 mV.

39. The reactor vessel of claim 1, further comprising a pH control system.

40. The reactor vessel of claim 39, wherein said pH control system further comprises:
a pH probe positioned at least partially within said interior space, said pH probe measuring a pH of a culture/media solution positioned in said interior portion;
a pH measurement unit operationally coupled to said pH probe, said pH measurement unit comparing an output of said pH probe to a predetermined pH upper value and a predetermined pH lower value; and
a pH adjustment unit, said pH adjustment unit injecting a pH buffer agent into said interior space when said pH measurement unit determines said output of said pH probe is at least trending towards said predetermined pH upper value, said pH adjustment unit injecting a second pH buffer agent into said interior space when said pH measurement unit determines said output of said pH probe is at least trending towards said predetermined pH lower value.

41. The reactor vessel of claim 40, wherein said first pH buffer agent comprises CO2.

42. The reactor vessel of claim 40, wherein said pH upper value is between 7.5 and 9.

43. The reactor vessel of claim 40, wherein said pH upper value is approximately 8.

44. The reactor vessel of claim 40, wherein said second pH buffer agent comprises sodium hydroxide.

45. The reactor vessel of claim 40, wherein said second pH buffer agent comprises potassium hydroxide.

46. The reactor vessel of claim 40, wherein said second pH buffer agent comprises calcium hydroxide.

47. The reactor vessel of claim 40, wherein said second pH buffer agent comprises sodium bicarbonate.

48. The reactor vessel of claim 40, wherein said second pH buffer agent comprises ammonia.

49. The reactor vessel of claim 40, wherein said second pH buffer agent comprises ammonium.

50. The reactor vessel of claim 40, wherein said second pH buffer agent comprises ammonia nitrate.

51. The reactor vessel of claim 40, wherein said pH lower value is between 6 and 8.

52. The reactor vessel of claim 40, wherein said pH lower value is approximately 7.6.

53. The reactor vessel of claim 1, further comprising an agitation system.

54. The reactor vessel of claim 53, wherein said agitation system further comprises:
an agitation drive means; and
an impeller operationally coupled to said agitation drive means, said impeller positioned within said reactor vessel.

55. The reactor vessel of claim 50, wherein said impeller rotates at between 1100 and 2100 rpm.

56. The reactor vessel of claim 54, wherein said impeller rotates at between 1500 and 1800 rpm.

57. The reactor vessel of claim 54, wherein said impeller rotates at greater than 110% of the resonance of the reactor vessel.

58. The reactor vessel of claim 54, wherein said impeller is a rushton impeller.

59. The reactor vessel of claim 54, wherein said agitation drive means further comprises:
a motor assembly;
a variable frequency drive control electrically coupled to said motor assembly for selectively controlling the speed of the motor assembly;
a magnetic coupling unit operationally coupled to said motor assembly and positioned at least partially within said interior space; and
an agitator shaft operationally coupled to said magnetic coupling unit, said agitator shaft being operationally coupled to said impeller.

60. The reactor vessel of claim 59, wherein said magnetic coupling unit is operationally coupled to said top wall.

61. The reactor vessel of claim 59, wherein said magnetic coupling unit is operationally coupled to said perimeter wall.

62. The reactor vessel of claim 1, wherein said interior space further comprises a culture/media holding space and a head space.

63. The reactor vessel of claim 62, wherein a volume of said culture/media holding space to a volume of said head space has a ratio between 1.5:1 to 5:1.

64. The reactor vessel of claim 62, wherein a volume of said culture/media holding space to a volume of said head space has a ratio of approximately 2.57:1.

65. The reactor vessel of claim 62, wherein said reactor vessel has an overall interior height between 10 and 220 feet.

66. The reactor vessel of claim 62, wherein said reactor vessel has an overall interior height between 60 and 150 feet.

67. The reactor vessel of claim 62, wherein said reactor vessel has an overall interior height of approximately 140 feet.

68. The reactor vessel of claim 62, wherein said reactor vessel has an overall interior volume between 75000 and 300000 gallons.

69. The reactor vessel of claim 62, wherein said reactor vessel has an overall interior volume of 250000 gallons.

70. The reactor vessel of claim 69, wherein said reactor vessel has an overall interior height of approximately 140 feet.

71. The reactor vessel of claim 70, wherein said reactor vessel has a volume of said culture/media holding space to a volume of said head space has a ratio of approximately 2.57:1.

72. The reactor vessel of claim 1, further comprising a culture/media recirculating system.

73. The reactor vessel of claim 72, wherein said culture media recirulating system further comprising:
a recirculation output port environmentally coupled to said interior space;
a recirulation pump operationally coupled to said recirculation output port; and
a recirculation input port environmentally coupled to said interior space, said recirculation input port operationally coupled to said recirculation pump.

74. Thee reactor vessel of claim 73, wherein said recirculation output port is operationally coupled to said perimeter wall.

75. The reactor vessel of claim 73, wherein said recirculation output port is operationally coupled to said top wall.

76. The reactor vessel of claim 73, wherein said recirculation input port is operationally coupled to said perimeter wall.

77. The reactor vessel of claim 1, wherein at least a portion of said perimeter wall has an insulating layer.

78. The reactor vessel of claim 1, wherein at least a portion of said insulating layer abuts an earthen wall.

79. The reactor vessel of claim 1, wherein at least a portion of said perimeter wall abuts an earthen wall.

80. The reactor vessel of claim 1, wherein said top wall comprises a floating roof.

81. The reactor vessel of claim 1, wherein said top wall comprises stainless steel.

82. The reactor vessel of claim 1, wherein said top wall comprises a polymeric membrane.

83. The reactor vessel of claim 1, wherein said top wall comprises fiberglass.

84. The reactor vessel of claim 1, wherein said top wall comprises concrete.

85. The reactor vessel of claim 84, wherein said top wall has an interior epoxy lining.

86. The reactor vessel of claim 84, wherein said top wall has an interior polymeric lining.

87. The reactor vessel of claim 84, wherein said top wall has an interior fiberglass lining.

88. The reactor vessel of claim 1, further comprising an input material stream port environmentally coupled to said interior space, said input material stream being for selectively routing an input material stream into said interior space.

89. The reactor vessel of claim 1, further comprising an output material stream port environmentally coupled to said interior space, said output material stream being for selectively routing an output material stream out of said interior space.

90. The reactor vessel of claim 89, further comprising a secondary vessel environmentally coupled to said output material stream port.

91. The reactor vessel of claim 90, wherein said secondary vessel further comprises a drain port.

92. The reactor vessel of claim 89, further comprising a culture conditioning chamber, said culture conditioning chamber being environmentally coupleable with said interior space.

93. The reactor vessel of claim 1, further comprising a biomass removal port environmentally coupled to said interior space.

94. The reactor vessel of claim 1, further comprising a culture/media input port environmentally coupled to said interior space.

95. The reactor vessel of claim 1, further comprising a culture sampling port operationally coupled to said interior space.

96. The reactor vessel of claim 1, further comprising a culture sampling port operationally coupled to a recirculation pipe.

97. A reactor vessel for the methanogenic conversion of an input material stream to an output materials stream, comprising:
a bottom wall;
a perimeter wall operationally coupled to said bottom wall and extending upwardly from said bottom wall;
a top wall operationally coupled to said perimeter wall;
said bottom wall, perimeter wall, and top wall defining an interior space environmentally separable from an exterior space outside of said reactor vessel;
at least one sparger, said being positioned substantially within said interior space, said at least one sparger being operationally coupled to an input gas stream;
a culture/media recirculating system;
wherein said culture media recirculating system further comprising:
a recirculation output port environmentally coupled to said interior space;
a recirculation pump operationally coupled to said recirculation output port;
a recirculation input port environmentally coupled to said interior space, said recirculation input port operationally coupled to said recirculation pump;
a recirculation pipe operationally coupled between said recirculation output port and said recirculation input port;
an oxidation reduction potential control system;
wherein said oxidation reduction potential control system further comprises:
An oxidation reduction potential probe positioned at least partially within recirculation pipe, said oxidation reduction potential probe measuring an oxidation reduction potential of a culture/media solution positioned in said interior portion;
An oxidation reduction potential measurement unit operationally coupled to said oxidation reduction potential probe, said oxidation reduction potential measurement unit comparing an output of said oxidation reduction potential probe to a at least one predetermined ORP value;
a pH control system;
wherein said pH control system further comprises:
an pH probe positioned at least partially within said recirculation pipe, said pH probe measuring a pH of a culture/media solution positioned in said interior portion;
an pH measurement unit operationally coupled to said pH probe, said pH measurement unit comparing an output of said pH probe to at least one predetermined pH value;
an agitation system;
wherein said agitation system further comprises:
an agitation drive means;
an impeller operationally coupled to said agitation drive means, said impeller positioned within said reactor vessel;
a thermal conditioning unit;
wherein said thermal conditioning unit further comprises:
a thermal transfer portion operationally coupled to said perimeter wall;
an thermal probe positioned at least partially within said recirculation pipe, said thermal probe measuring a temperature of a culture/media solution positioned in said interior portion; and
an thermal measurement unit operationally coupled to said thermal probe, said thermal measurement unit comparing an output of said thermal probe to at least one predetermined thermal value.

98. The reactor vessel of claim 99, further comprising an input material stream port environmentally coupled to said interior space, said input material stream being for selectively routing an input material stream into said interior space.

99. The reactor vessel of claim 99, further comprising an output material stream port environmentally coupled to said interior space, said output material stream being for selectively routing an output material stream out of said interior space.

100. The reactor vessel of claim 99, further comprising a secondary vessel environmentally coupled to said output material stream port.

101. The reactor vessel of claim 100, wherein said secondary vessel further comprises a drain port.

102. The reactor vessel of claim 97, further comprising a biomass removal port environmentally coupled to said interior space.

103. The reactor vessel of claim 97, further comprising a culture/media input port environmentally coupled to said interior space.

104. The reactor vessel of claim 97, further comprising a culture sampling port operationally coupled to said interior space.

105. The reactor vessel of claim 97, further comprising a culture sampling port operationally coupled to said recirculation pipe.

106. The reactor vessel of claim 97, wherein said interior space further comprises a culture/media holding space and a head space.

107. The reactor vessel of claim 106, wherein a volume of said culture/media holding space to a volume of said head space has a ratio between 1.5:1 to 5:1.

108. The reactor vessel of claim 106, wherein a volume of said culture/media holding space to a volume of said head space has a ratio of approximately 2.57:1.

109. The reactor vessel of claim 106, wherein said reactor vessel has an overall interior height between 10 and 220 feet.

110. The reactor vessel of claim 106, wherein said reactor vessel has an overall interior height between 60 and 150 feet.

111. The reactor vessel of claim 106, wherein said reactor vessel has an overall interior height of approximately 140 feet.

112. The reactor vessel of claim 106, wherein said reactor vessel has an overall interior volume between 75000 and 300000 gallons.

113. The reactor vessel of claim 106, wherein said reactor vessel has an overall interior volume of 250000 gallons.

114. The reactor vessel of claim 113, wherein said reactor vessel has an overall interior height of approximately 140 feet.

115. The reactor vessel of claim 114, wherein said reactor vessel has a volume of said culture/media holding space to a volume of said head space has a ratio of approximately 2.57:1.

116. The reactor vessel of claim 97, wherein said reactor vessel further comprises a data system operationally coupled to at least one of said culture media recirculation system, oxidation reduction potential control system, pH control system, agitation system, thermal conditioning unit, or at least one sparger.

117. The reactor vessel of claim 97, wherein said reactor vessel further comprises a hydrogen diffuser system positioned substantially within said interior space, said hydrogen differ system being for releasing hydrogen from a mixed gas stream flowing through said hydrogen diffuser system into said interior space.

118. The reactor vessel of claim 117, wherein said hydrogen diffuser system is operationally coupled between an input material stream port and a first output material stream port.

119. The reactor vessel of claim 118, wherein said first output material stream port is operationally coupled to an input of an intermediate processing unit, said intermediate processing unit providing a filtering function, said intermediate processing unit having an output operationally coupled to a second input material stream port environmentally coupled to said interior space.

120. The reactor vessel of claim 117, wherein said hydrogen diffuser system is operationally coupled between a first output material stream port and a second output material stream port.

121. The reactor vessel of claim 117, wherein said hydrogen diffuser system further comprises a length of tubing having a perimeter wall permeable by hydrogen and substantially impermeable to other components in said mixed gas stream.

122. A reactor vessel for the methanogenic conversion of an input material stream to an output materials stream, comprising:
a bottom wall;
a perimeter wall operationally coupled to said bottom wall and extending upwardly from said bottom wall;
a top wall operationally coupled to said perimeter wall;
said bottom wall, perimeter wall, and top wall defining an interior space environmentally separable from an exterior space outside of said reactor vessel;
at least one sparger, said being positioned substantially within said interior space, said at least one sparger being operationally coupled to an input gas stream;
a hydrogen diffuser system positioned substantially within said interior space, said hydrogen differ system being for releasing hydrogen from a mixed gas stream flowing through said hydrogen diffuser system into said interior space; and
wherein said hydrogen diffuser system is operationally coupled between a first output material stream port and a second output material stream port.

123. The reactor vessel of claim 122, wherein said interior space further comprises a plurality of zones.

124. The reactor vessel of claim 123, wherein said plurality of zones further comprises a tank pressure zone, said tank pressure zone having a greater pressure due to the column of culture/media within and above said tank pressure zone.

125 The reactor vessel of claim 123, wherein said plurality of zones further comprises a diffuser zone, said hydrogen diffuser system being positioned substantially within said diffuser zone.

126. The reactor vessel of claim 123, wherein said plurality of zones further comprises an agitation and defoaming zone.

127. The reactor vessel of claim 123, wherein said plurality of zones further comprises:
a tank pressure zone, said tank pressure zone having a greater pressure due to the column of culture/media within and above said tank pressure zone;
a diffuser zone, said hydrogen diffuser system being positioned substantially within said diffuser zone, said diffuser zone being positioned vertically above said tank pressure zone; and
an agitation and defoaming zone positioned vertically above said diffuser zone.

128. The reactor vessel of claim 127, wherein said interior space is adapted for holding a vertical column of media/culture of at least 50 feet.

129. The reactor vessel of claim 127, wherein said interior space is adapted for holding a vertical column of media/culture of at least 100 feet.

130. The reactor vessel of claim 127, further comprising a defoaming bar positioned substantially within said agitation and defoaming zone.

131. The reactor vessel of claim 127, further comprising a defoaming agent input port environmentally coupled with said interior space, said defoaming agent input port being for the selective introduction of a defoaming agent into said interior space.
